SEQUENCE LISTING (5 sequences)

SEQ ID NO: 1 - protein, 635 aa, Homo sapiens
  REGION 1..635: note = Thrombopoietin receptor (TPOR)
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 2 - protein, 96 aa, Homo sapiens
  REGION 1..96: note = TPOR amino acids 26-121 (D1)
QDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRT

SEQ ID NO: 3 - protein, 258 aa, Homo sapiens
  REGION 1..258: note = TPOR amino acids 26-283 (D1D2)
QDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGD

SEQ ID NO: 4 - protein, 465 aa, Homo sapiens
  REGION 1..465: note = TPOR amino acids 26-490 (D1D2D3D4)
QDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETA

SEQ ID NO: 5 - protein, 350 aa, Homo sapiens
  REGION 1..350: note = Thrombopoietin (TPO)
MELTELLLVVMLLLTARLTLSSPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQLPPQGRTTAHKDPNAIFLSFQHLLRGKVRFLMLVGGSTLCVRRAPPTTAVPSRTSLVLTLNELPNRTSGLLETNFTASARTTGSGLLKWQQGFRAKIPGLLNQTSRSLDQIPGYLNRIHELLNGTRGLFPGPSRRTLGAPDISSGTSDTGSLPPNLQPGYSPSPTHPPTGQYTLFPLPPTLPTPVVQLHPLLPDPSAPTPTPTSPLLNTSYTHSQNLS

(19) United States
(12) Patent Application Publication
(10) Pub. No.: US 2025/0241986 A1
(43) Pub. Date: Jul. 31, 2025

(54) SOLUBLE THROMBOPOIETIN RECEPTOR FRAGMENTS AND USES THEREOF

(71) Applicant: UNIVERSITE CATHOLIQUE DE LOUVAIN, Louvain la Neuve (BE)

(72) Inventors: Stefan CONSTANTINESCU, Bruxelles (BE); Audrey NEDELEC, Woluwe-Saint-Lambert (BE); Nicolas PAPADOPOULOS, Bruxelles (BE)

(73) Assignee: UNIVERSITE CATHOLIQUE DE LOUVAIN, Louvain la Neuve (BE)

(21) Appl. No.: 18/853,266

(22) PCT Filed: Mar. 31, 2023
(86) PCT No.: PCT/EP2023/058573

§ 371 Date: Oct. 1, 2024

(30) Foreign Application Priority Data
Apr. 1, 2022 (EP) 22166303.2

(51) Int. Cl. A61K 38/17; A61K 47/68; C07K 14/715

(52) U.S. Cl. CPC A61K 38/1793; A61K 47/6811; C07K 14/715; C07K 2319/30

(57) ABSTRACT

A polypeptide including the extracellular domain of the thrombopoietin receptor or fragments thereof, and its use as a medicament. Also, the treatment and/or prevention of thrombopoietin-related diseases, in particular myeloproliferative neoplasms, in which the polypeptide, a fusion protein including the polypeptide, a nucleic acid including a sequence coding for the polypeptide, or a vector including the nucleic acid is administered to a subject in need thereof.

### FIELD OF INVENTION

The present invention relates to a polypeptide comprising the extracellular domain of the thrombopoietin receptor or fragments thereof, and its use as a medicament. The present invention also relates to the treatment and/or prevention of thrombopoietin-related diseases, in particular myeloproliferative neoplasms.

### BACKGROUND OF INVENTION

Myeloproliferative Neoplasms (MPNs) are malignant blood diseases of aged individuals, and represent a significant burden for the healthcare systems. MPNs can evolve to a very severe condition called secondary Acute Myeloid Leukemia (AML), which is almost always fatal. The three major MPNs types, namely Polycythemia Vera (PV), Essential Thromocythemia (ET) and Myelofibrosis (MF) can occur with an incidence that can reach 1/5,000 persons per year.

In all three diseases, acquired mutations in the hematopoietic stem cells (HSCs) lead to pathological activation of the TPOR/JAK2 signaling that drives the pathology. The JAK2 V617F mutation is responsible for 70% of all MPNs and is present in over 96% of PV and 60% of ET and MF cases, and leads to an over-activation of the JAK2-STAT5 pathway via the thrombopoietin receptor (TPOR) and, to a lesser extent, other cytokine receptors (EpoR, G-CSFR).

Several studies demonstrated the requirement of thrombopoietin (THPO), for the development of MPN pathology independently of the driver mutation. A recent study notably showed that THPO inhibition led to significant improvement of the disease in mouse models.

Non-specific chemotherapy, radiation therapy and bone marrow transplants are common strategies for the treatment of malignant blood diseases. However, these therapeutic approaches are invasive and heavy for patients. Therefore, there is still a growing need in this field to bring new resources or tools improving therapeutic approaches.

Previous studies identified binding sites of THPO to TPOR (Chen Wei-Min et al., 2010). A method of producing the soluble fragment of TPOR has also been described (Zhang Qing et al., 2004).

Chachoua et al., 2016 discloses the use of D1-D2 fragment region of TPOR inhibiting the TPOR dependent signaling pathway, but through the scavenging of intracellular CALR mutants' activity (“cis” action), and not of THPO. These models are not suitable for clinical uses as they all acts intracellularly thereby requiring gene transfer approach. The present invention aims at using the extracellular domain of the receptor TPOR coupled to the Fc fragment of an IgG1 as a trap molecule for circulating thrombopoietin (THPO). This approach aims at competition or partial blocking of the abnormally elevated TPOR induced signaling which is critical for the development of all types of MPNs. A novel, potent and specific THPO inhibitor would thus provide a much-needed improvement for the treatment and/or prevention of MPNs.

In addition to MPNs, several hereditary and sporadic conditions are due to a hyperactivation of TPOR signaling. In hereditary thrombocytosis, germline mutations in TPOR or JAK2 lead to cytokine independent signaling. Such pathologies are not clonal but lead to complications and need to be addressed early. In another condition, mutations in non-translated regions lead to hyperproduction of THPO ligand by the liver. This type of pathology would also strongly benefit of THPO inhibitor. In de novo AML, studies also suggested that high levels of THPO signaling may promote oncogenic signaling. Finally, infections and inflammations may lead to higher THPO production by several tissues resulting in thrombocytosis. For all these pathologies, a THPO inhibitor would be strongly useful.

### SUMMARY

The present invention relates to a polypeptide comprising an amino acid sequence having at least 75% sequence identity with SEQ ID NO: 2 or a variant thereof that binds to thrombopoietin (THPO). In one embodiment, this polypeptide is for use in the treatment and/or prevention of a disease.

In some embodiments, the polypeptide is able to inhibit, reduce, prevent and/or disturb the binding of THPO to its receptor. In some embodiments, the polypeptide is able to inhibit, reduce, prevent and/or disturb the TPOR signaling pathway or TPOR-dependent signaling pathway.

In some embodiments, the amino acid sequence of the polypeptide has at least 75% sequence identity with SEQ ID NO: 3.

In some embodiments, the amino acid sequence of the polypeptide has at least 75% sequence identity with SEQ ID NO: 4.

The present invention further relates to a fusion protein comprising the polypeptide of the invention. In one embodiment, this fusion protein is for use in the treatment and/or prevention of a disease.

In one embodiment, the fusion protein further comprises a second polypeptide that increases stability and/or decreases immunogenicity of the first polypeptide, preferably a Fc region of an immunoglobulin or a functional equivalent thereof.

In one embodiment, the immunoglobulin is IgA, IgD, IgE, IgG, or IgM, preferably IgG.

The present invention further relates to a nucleic acid comprising a sequence encoding the polypeptide of the invention or fusion protein comprising said polypeptide. In one embodiment, this nucleic acid is for use in the treatment and/or prevention of a disease.

The present invention further relates to a vector comprising a nucleic acid encoding the polypeptide of the invention or fusion protein comprising said polypeptide. In one embodiment, this vector is for use in the treatment and/or prevention of a disease.

The present invention further relates to a composition comprising the polypeptide, fusion protein, nucleic acid according, or vector according to the invention, and an acceptable vehicle. In one embodiment, this composition is for use in the treatment and/or prevention of a disease.

The present invention further relates to a pharmaceutical composition comprising (i) the polypeptide, fusion protein, nucleic acid according, or vector according to the invention, and (ii) at least one pharmaceutically acceptable vehicle. In one embodiment, this pharmaceutical composition is for use in the treatment and/or prevention of a disease.

The present invention further relates to the polypeptide, fusion protein or pharmaceutical composition according to the invention, for use as a medicament.

The present invention further relates to the polypeptide, fusion protein or pharmaceutical composition according to the invention, for use in the treatment and/or prevention of thrombopoietin-related diseases.

In some embodiments, thrombopoietin-related diseases are disease, preferably inflammations and/or infections, where signaling of the thrombopoietin receptor is increased. Increased signaling can occur due to increased levels of thrombopoietin or due to potentiation by thrombopoietin of pathologic signaling, even in the absence of increased thrombopoietin levels.

In some embodiments, thrombopoietin-related diseases are selected from the group comprising or consisting of hematological diseases, chronic liver diseases, thrombopoietin-related infections, thrombopoietin-related inflammations and hereditary thrombocytosis, preferably selected from the group comprising or consisting of myeloproliferative neoplasms (MPN), hereditary thrombocytosis, and de novo AML (Acute Myeloid Leukemia).

In some embodiments, thrombopoietin-related diseases are myeloproliferative neoplasms (MPN).

The present invention further relates to a kit for treating and/or preventing thrombopoietin-related diseases comprising (i) a polypeptide, fusion protein, or pharmaceutical composition, and (ii) means to administer said means to administer said polypeptide, fusion protein or pharmaceutical composition, optionally further comprising an anticancer agent.

### Definitions

The term “amino acid substitution” refers to the replacement in a polypeptide of one amino acid with another amino acid. In one embodiment, an amino acid is replaced with another amino acid having similar structural and/or chemical properties, e.g., conservative amino acid replacements. “Conservative amino acid substitution” may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; negatively charged (acidic) amino acids include aspartic acid and glutamic acid. Non-conservative substitutions will entail exchanging a member of one of these classes for another class. For example, amino acid substitutions can also result in replacing one amino acid with another amino acid having different structural and/or chemical properties, for example, replacing an amino acid from one group (e.g., polar) with another amino acid from a different group (e.g., basic). Amino acid substitutions can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

The term “identity” refers to a measure of the identity of nucleotide sequences or amino acid sequences. In general, the sequences are aligned so that the highest order match is obtained. “Identity” per se has an art-recognized meaning and can be calculated using published techniques. See, e.g.: Computational Molecular Biology, Lesk, A. M., ed., Oxford University Press, New York, 1988; Biocomputing: Informatics And Genome Projects, Smith, D. W., ed., Academic Press, New York, 1993; Computer Analysis Of Sequence Data, Part I, Griffin, A. M., and Griffin, H. G., eds, Humana Press, New Jersey, 1994; Sequence Analysis In Molecular Biology, von Heijne, G., Academic Press, 1987; and Sequence Analysis Primer, Gribskov, M. and Devereux, J., eds, M Stockton Press, New York, 1991. While there exist a number of methods to measure identity between two polynucleotide or polypeptide sequences, the term “identity” is well known to skilled artisans (Carillo and Lipton, SIAM J Applied Math, 1998, 48:1073). Methods commonly employed to determine identity or similarity between two sequences include, but are not limited to, those disclosed in Guide to Huge Computers, Martin J. Bishop, ed., Academic Press, San Diego, 1994; and Carillo and Lipton, SIAM J Applied Math, 1998, 48:1073. Methods to determine identity and similarity are codified in computer programs. Computer program methods to determine identity and similarity between two sequences include, but are not limited to, GCG program package (Devereux et al., J Molec Biol, 1990, 215:403), the GAP program. As an illustration, by a polynucleotide having a nucleotide sequence having at least, for example, 95% “identity” to a reference nucleotide sequence is intended that the nucleotide sequence of the polynucleotide is identical to the reference sequence except that the polynucleotide sequence may include an average up to five point-mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

The term “peptide linker”, or “linker” also called “spacer peptide”, refers to a peptide used to link 2 peptides or polypeptides together. In one embodiment, a peptide linker of the invention comprises from 3 to 50 amino acids. Peptide linkers are known in the art or are described herein.

The term “pharmaceutically acceptable excipient” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. A pharmaceutically acceptable carrier or excipient refers to a non-toxic solid, semi-solid or liquid filler, diluent, encapsulating material or formulation auxiliary of any type. For human administration, preparations should meet sterility, pyrogenicity, general safety and purity standards as required by FDA Office of Biologics standards.

The term “nucleic acid” or “polynucleotide” refers to any polyribonucleotide or polydeoxyribonucleotide, which may be unmodified RNA or DNA or modified RNA or DNA. “Nucleic acid” or “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “Nucleic acid” or “polynucleotide” refers to triple-stranded regions comprising RNA or DNA or both RNA and DNA. The term “nucleic acid” or “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “nucleic acid” or “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

The term “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

The term “protein” refers to a sequence of more than 100 amino acids and/or to a multimeric entity. The proteins of the invention are not limited to a specific length of the product. The term “polypeptide” or “protein” does not refer to or exclude post-expression modifications of the protein, for example, glycosylation, acetylation, phosphorylation and the like, as well as other modifications known in the art, both naturally occurring and non-naturally occurring. Such modifications are well described in basic texts and in more detailed monographs, as well as in a voluminous research literature. Modifications can occur anywhere in a polypeptide or protein, including the peptide backbone, the amino acid side-chains and the amino or carboxyl termini. It will be appreciated that the same type of modification may be present in the same or varying degrees at several sites in a given polypeptide or protein. Also, a given polypeptide or protein may contain many types of modifications. Polypeptides or proteins may be branched as a result of ubiquitination, and they may be cyclic, with or without branching. Cyclic, branched and branched cyclic polypeptides or proteins may result from posttranslational natural processes or may be made by synthetic methods. Modifications include acetylation, acylation, ADP-ribosylation, amidation, covalent attachment of flavin, covalent attachment of a hem moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-linkings, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino of amino acids to proteins such as arginylation, and ubiquitination. See, for instance, “Proteins-structure and molecular properties”, 2nd Ed., T. E. Creighton, W. H. Freeman and Comany, New York, 1993; Wolt, F., “Posttranslational Protein Modifications: Perspectives and Prospects”, Posttranslational covalent modification of proteins, B. C. Johnson, Ed., Academic Press, New York, 1983, pgs. 1-12; Seifter et al., “Analysis for protein modifications and nonprotein cofactors”, Meth Enzymol, 1990, 182:626-646; Rattan et al, “Protein Synthesis: Posttranslational Modifications and Aging”, Ann NY Acad Sci, 1992, 663:48-62. A protein may be an entire protein, or a subsequence thereof.

The term “fusion protein” refers to a protein having at least two heterologous polypeptides covalently linked either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion proteins may be fused in any order. This term also refers to conservatively modified variants, polymorphic variants, alleles, mutants, subsequences, and interspecies homologs of the antigens that make up the fusion protein.

The term “fused” refers to components that are linked by peptide bonds, either directly or through one or more peptide linkers.

The term “immunoglobulin” includes a protein having a combination of two heavy and two light chains, whether or not it possesses any relevant specific immunoreactivity.

The term “treating” or “treatment” or “alleviation” refers to both therapeutic treatment and prophylactic or preventative measures; wherein the object is to prevent or slow down (lessen) the disease or condition, preferably thrombopoietin-related disease, more preferably MPN. Those in need of treatment include those already affected with the disease or condition, preferably thrombopoietin-related disease, more preferably MPN, as well as those prone to have the disease or condition, preferably thrombopoietin-related disease, more preferably MPN, or those in whom the disease or condition, preferably thrombopoietin-related disease, more preferably MPN, is to be prevented. A subject or mammal is successfully “treated” for the disease or condition, preferably thrombopoietin-related disease, more preferably MPN, if, after receiving a therapeutic amount of a polypeptide or fusion protein according to the methods of the present invention, the patient shows observable and/or measurable reduction in or absence of one or more of the following: reduction in the number of pathogenic cells; reduction in the percent of total cells that are pathogenic; and/or relief to some extent, one or more of the symptoms associated with the disease or condition, preferably thrombopoietin-related disease, more preferably MPN; reduced morbidity and mortality, and improvement in quality of life issues. The above parameters for assessing successful treatment and improvement in the disease or condition, preferably thrombopoietin-related disease, more preferably MPN, are readily measurable by routine procedures familiar to a physician.

The term “variant” refers to a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions identical. A variant and reference polypeptide may differ in amino acid sequence by one or more substitutions (preferably conservative), additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. Variants should retain one or more of the biological activities of the reference polypeptide.

### DETAILED DESCRIPTION

Thrombopoietin receptor, herein referred to as TPOR, is a transmembrane protein mostly expressed at the plasma membrane of megakaryocytes, platelets, hemangioblasts, and hematopoietic stem cells, that triggers the JAK/STAT signaling pathway upon binding to its ligand thrombopoietin. Abnormal TPOR activation is associated with pathologic conditions, including myeloproliferative neoplasms. In the present disclosure, the full amino acid sequence of TPOR is referred to as SEQ ID NO: 1 (corresponding to UniProt ID P40238).

TPOR is a 635 amino acids long protein that has three functional domains: an extracellular domain (ECD) comprising the thrombopoietin binding site, a transmembrane domain (TMD), and a cytoplasmic/intracellular domain (ICD). In particular, the ECD is divided into 4 subdomains, namely D1, D2, D3 and D4.

Thrombopoietin is referred herein to as THPO. However, thrombopoietin may also be named TPO, MPL ligand or megakaryocyte colony-stimulating factor.

The present invention relates to a polypeptide comprising an amino acid sequence having at least 75% sequence identity with SEQ ID NO: 2 or a variant thereof that binds to thrombopoietin (THPO).

SEQ ID NO: 2 relates to the sequence of the subdomain D1 of the ECD (hereinafter referred to as D1).

In one embodiment, the polypeptide of the invention is an isolated polypeptide.

In one embodiment, the polypeptide of the invention is capable of preventing, inhibiting, reducing or disturbing the binding of thrombopoietin (THPO) to its receptor (TPOR/MPL).

In one embodiment, the amino acid sequence of the polypeptide of the invention does not comprise SEQ ID NO: 1.

In some embodiments, the polypeptide of the invention comprises at least 96, 97, 98, 99, 100, 101, 102, 103, 104, 105, 106, 107, 108, 109, or 110 amino acids. In some embodiments, the polypeptide of the invention comprises at least 258, 259, 260, 261, 262, 263, 264, 265, 266, 267, 268, 269, or 270 amino acids. In some embodiments, the polypeptide of the invention comprises at least 465, 466, 467, 468, 469, 470, 471, 472, 473, 474, 475, 476, 477, 478, 479, or 480 amino acids.

In some embodiments, the polypeptide of the invention comprises from 96 to 465 amino acids, or from 96 to 258 amino acids. In some embodiments, the polypeptide of the invention comprises from 96 to 500 amino acids, from 96 to 400 amino acids, from 96 to 300 amino acids, or from 96 to 200 amino acids.

In some embodiments, the polypeptide of the invention comprises from 258 to 465 amino acids. In some embodiments, the polypeptide of the invention comprises from 258 to 500 amino acids, from 258 to 400 amino acids, or from 258 to 300 amino acids.

In some embodiments, the polypeptide of the invention comprises from 465 to 500 amino acids.

In some embodiments, the polypeptide of the invention comprises at most 635 amino acids. In one embodiment, the polypeptide comprises at most 634 amino acids.

In one embodiment, the polypeptide of the invention comprises at most 600, 500, 400, 300, or 200 amino acids.

In one embodiment, the polypeptide of the invention comprises an amino acid sequence having at least 80% sequence identity with SEQ ID NO: 2, preferably at least 85%, 90%, 95%, 96%, 97%, 98%, 99% or 100% identical. In one embodiment, the amino acid sequence of the polypeptide of the invention comprises or consists of SEQ ID NO: 2.

In one embodiment, the polypeptide of the invention comprises an amino acid sequence having at least 75% sequence identity with SEQ ID NO: 3, preferably at least 80%, 85%, 90%, 95%, 96%, 97%, 98%, 99% or 100% identical. In one embodiment, the amino acid sequence of the polypeptide of the invention comprises or consists of SEQ ID NO: 3. In one embodiment, the amino acid sequence of the polypeptide of the invention does not consist of SEQ ID NO: 3.

SEQ ID NO: 3 relates to the sequence of the subdomains D1 and D2 of the ECD (hereinafter referred to as D1D2).

In one embodiment, the polypeptide of the invention comprises an amino acid sequence having at least 75% sequence identity with SEQ ID NO: 4, preferably at least 80%, 85%, 90%, 95%, 96%, 97%, 98%, 99% or 100% identical. In one embodiment, the amino acid sequence of the polypeptide of the invention comprises or consists of SEQ ID NO: 4.

SEQ ID NO: 4 relates to the sequence of the subdomains D1 to D4 of the ECD (hereinafter referred to as D1D2D3D4).

In some embodiments, the polypeptide of the invention can act intracellularly (“cis” action) or extracellularly (“trans” action).

In a preferred embodiment, the polypeptide of the invention acts extracellularly (“trans” action).

In some embodiments, the polypeptide of the invention is soluble. Advantageously, the solubility of the polypeptide of the invention allows that it can act extracellularly (“trans” action).

In some embodiments, the amino acid sequence of the polypeptide of the invention comprises at least one asparagine residue (Asn) at positions selected from the group comprising or consisting of positions 117, 178, 298 and 358, wherein said positions are defined with respect to the amino acid sequence SEQ ID NO: 1. As used herein, at least one Asn residue means 1, 2, 3 or 4 Asn residues. In some embodiments, the amino acid sequence of the polypeptide of the invention comprises Asn residues at positions 117, 178, 298 and 358, wherein said positions are defined with respect to the amino acid sequence SEQ ID NO: 1.

In some embodiments, the Asn residues are modified. In some embodiments, the Asn residues are glycosylated. In one embodiment, glycan(s) are mature. Non limitative examples of mature glycosylation include Hex(5)HexNAc(2)dHex(1) and HexNAc(2)Hex(3)Fuc(1)Sias(1). In another embodiment, glycan(s) are immature. Non limitative examples of immature glycosylations include HexNAc(2)Hex(10), HexNAc(2)Hex(9) and HexNAc(2)Hex(8). In some embodiments, the Asn residues are phosphorylated, glycosylated, ubiquitinated, nitrosylated, methylated, acetylated or lipidated.

In some embodiments, at least one Asn residue is not modified. In some embodiments, the Asn residues are not modified. In some embodiments, at least one Asn residue is not glycosylated. In some embodiments, the Asn residues are not glycosylated.

In some embodiments, the polypeptide of the invention binds to thrombopoietin (THPO) of SEQ ID NO: 5, or variants thereof.

In some embodiments, the amino acid sequence of the polypeptide of the invention comprises at least one mutation compared to the amino acid sequence of SEQ ID NO: 2, wherein said mutation increases the affinity of the polypeptide for THPO or variants thereof.

As used herein, the term “mutation” refers to insertion(s), deletion(s), truncation(s) or substitution(s) of at least one amino acid by at least one natural and/or non-natural different amino acid. Non limitative examples of substitutions of amino acid with different chemical properties include the substitution of a positively charged arginine with a negatively charged glutamic acid, or the substitution of a polar asparagine with a non-polar tryptophan.

As used herein, the term “affinity” refers to the “binding affinity” between two molecules, herein preferably proteins or polypeptides. The binding affinity is typically assessed by determining the dissociation constant (Kd) or, for example, measuring the kinetics of a reaction (Michaelis constant, Km) using methods known in the art. In particular, when measuring the effects of an inhibitor, e.g., a competitive inhibitor or a non-competitive inhibitor, the inhibition constant (Ki) can be calculated.

In some embodiments, the polypeptide of the invention, in particular the fragment region D1D2D3D4, has a better interaction with THPO than the fragment region D1D2. Therefore, the polypeptide of the invention is more efficient to compete against THPO than the fragment region D1D2.

In one embodiment, the polypeptide is for use as a medicament. In one embodiment, the polypeptide is for use treating and/or preventing a disease.

The invention further relates to a fusion protein comprising a polypeptide as described hereinabove and at least one other polypeptide. As used herein in the context of the fusion protein, the polypeptide of the invention is referred to as “the first polypeptide” and the at least one other polypeptide is referred to as “the second polypeptide”.

In some embodiments, the second polypeptide increases stability of the first polypeptide. By increasing stability, it is meant the improvement of the resistance of the polypeptide to modifications or degradations, including but not limited to lysis, truncation, irreversible post-translational modifications or unfolding. In practice, the increase of stability results in an increase of the half-life of the polypeptide within the organism.

Methods for measuring protein or peptide stability are known in the art and include, without limitation, differential scanning calorimetry (DSC), bleach-chase, cycloheximide-chase, circular dichroism (CD) spectroscopy, SDS-PAGE electrophoresis.

In some embodiments, the second polypeptide decreases immunogenicity of the first polypeptide. By immunogenicity, it is meant the potency to trigger a detectable immune response within an organism, e.g., the production of antibodies, B cells, helper T cells, and/or cytotoxic T cells, directed specifically to one or more antigen.

Methods for measuring a T cell immune response are well known by the skilled artisan and include, without limitation, monitoring the production of IFN-gamma.

In one embodiment, the second polypeptide of the fusion protein is a Fc region of an immunoglobulin or a functional equivalent thereof. By Fc fragment of an immunoglobulin, it is meant the carboxy-terminal portions of both H chains held together by disulfides.

In some embodiments, the second polypeptide of the fusion protein is a Fc region of an immunoglobulin selected from the group comprising or consisting of IgA, IgD, IgE, IgG, IgM, and functional equivalents thereof.

In a preferred embodiment, the immunoglobulin is IgG, preferably IgG1 or IgG2. In another embodiment, the immunoglobulin is IgA. In another embodiment, the immunoglobulin is IgD. In another embodiment, the immunoglobulin is IgE. In another embodiment, the immunoglobulin is IgM.

In a preferred embodiment, the immunoglobulin is a human immunoglobulin. In another embodiment, the immunoglobulin is a non-human immunoglobulin.

In some embodiments, the immunoglobulin is modified, e.g., genetically and/or post-translationally modified. In practice, this modification is for increasing the stability of the fusion protein and/or its immunogenicity.

In one embodiment, the polypeptides of the fusion protein of the invention are disposed in a single, contiguous polypeptide chain.

In one embodiment, the fusion protein of the invention further comprises at least one peptide linker. In one embodiment, the polypeptides of the fusion protein of the invention are linked to each other through one or more peptide linkers.

Methods which are well known to those skilled in the art can be used to construct expression vectors containing the coding sequence of a fusion protein (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the fusion protein (fragment) (i.e., the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g., on a single vector, or in separate polynucleotide constructs, e.g., on separate (different) vectors. Furthermore, any vector may contain a single coding region, or may comprise two or more coding regions, e.g., a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the fusion protein (fragment) of the invention, or variant or derivative thereof. Heterologous coding regions include without limitation specialized elements or motifs, such as a secretory signal peptide or a heterologous functional domain. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g., the immediate early promoter, in conjunction with intron-A), simian virus 40 (e.g., the early promoter), and retroviruses (such as, e.g., Rous sarcoma virus). Other transcription control regions include those derived from vertebrate genes such as actin, heat shock protein, bovine growth hormone and rabbit a-globin, as well as other sequences capable of controlling gene expression in eukaryotic cells. Additional suitable transcription control regions include tissue-specific promoters and enhancers as well as inducible promoters (e.g., promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Fusion proteins prepared as described herein may be purified by art-known techniques such as high-performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the fusion protein binds. The purity of the fusion protein can be determined by any of a variety of well-known analytical methods including gel electrophoresis, high pressure liquid chromatography, and the like.

In one embodiment, the fusion protein as described hereinabove is soluble. In some embodiments, the second polypeptide increases the solubility of the first polypeptide.

In one embodiment, the fusion protein as described hereinabove is for use as a medicament.

The invention also relates to a nucleic acid comprising a sequence encoding the polypeptide or the fusion protein as described hereinabove.

In one embodiment, the nucleic acid encoding the fusion protein of the invention may be expressed as a single nucleic acid molecule that encodes the entire fusion protein or as multiple (e.g., two or more) nucleic acid molecules that are co-expressed. Polypeptides encoded by nucleic acid molecules that are co-expressed may associate through, e.g., disulfide bonds or other means, to form a functional fusion protein.

In one embodiment, the nucleic acid molecule is DNA. In another embodiment, the nucleic acid molecule is RNA, for example, in the form of messenger RNA (mRNA).

In one embodiment, the nucleic acid is linear. In another embodiment, the nucleic acid is circular.

In one embodiment, the nucleic acid as described hereinabove is for use as a medicament.

Another object of the invention is a vector comprising a nucleic acid according the invention. In some embodiments, at least one nucleic acid molecule is comprised in the one vector.

Within the scope of the instant invention, the expression “at least one nucleic acid” is intended to include 1, 2, 3, 4, 5, 6, 7, 8, 9, 10, 15, 20, 25, 30, 35, 40, 45, 50 or more nucleic acid molecules.

In some embodiments, the vector allows the controlled expression of at least one polypeptide and/or fusion protein. As used herein, the expression “controlled expression” is intended to refer to an expression that is controlled in time and/or space. In other words, the controlled expression of the polypeptide according to the invention may occur in a specific location of the body, such as, e.g., a specific organ, and/or for a specific time period.

In certain embodiments, the vector is a viral vector. Non limitative examples of viral vectors include adenovirus, adeno-associated virus (AAV), alphavirus, herpesvirus, lentivirus, non-integrative lentivirus, retrovirus, vaccinia virus and baculovirus.

In certain embodiments, the vector is a non-viral vector. Non limitative examples of non-viral vectors include inorganic particles (e.g., gold, calcium phosphate), lipidic emulsions, lipidic nanoparticles (e.g., liposomes), DNA-binding protein or peptide.

In one embodiment, the vector as described hereinabove is for use as a medicament.

The invention further relates to a composition comprising a polypeptide, a fusion protein, a nucleic acid or a vector according to the invention, and an acceptable vehicle.

The invention further relates to a pharmaceutical composition comprising a polypeptide, a fusion protein, a nucleic acid or a vector according to the invention, and a pharmaceutically acceptable vehicle.

In some embodiments, the pharmaceutically acceptable vehicle is selected in a group comprising or consisting of a solvent, a diluent, a carrier, an excipient, a dispersion medium, a coating, an antibacterial agent, an antifungal agent, an isotonic agent, an absorption delaying agent and any combinations thereof. The carrier, diluent, solvent or excipient must be “acceptable” in the sense of being compatible with the polypeptide, or derivative thereof, and not be deleterious upon being administered to an individual. Typically, the vehicle does not produce an adverse, allergic or other untoward reaction when administered to an individual, preferably a human individual.

For the particular purpose of human administration, the pharmaceutical compositions should meet sterility, pyrogenicity, general safety and purity standards as required by regulatory offices, such as, for example, the Food and Drugs Administration (FDA) Office or the European Medicines Agency (EMA).

In some embodiments, the carrier may be water or saline (e.g., physiological saline), which will be sterile and pyrogen free. Suitable excipients include mannitol, dextrose, lactose, starch, magnesium stearate, sodium saccharine, cellulose, magnesium carbonate, and the like.

Acceptable carriers, solvents, diluents and excipients for therapeutic use are well known in the pharmaceutical art, and are described, for example, in Remington's Pharmaceutical Sciences, Mack Publishing Co. (A. R. Gennaro ed. 1985). The choice of a suitable pharmaceutical carrier, solvent, excipient or diluent can be made with regard to the intended route of administration and standard pharmaceutical practice. The pharmaceutical compositions may comprise as, or in addition to, the carrier, excipient, solvent or diluent any suitable binder, lubricant, suspending agent, coating agent, or solubilizing agent. Preservatives, stabilizers, dyes and even flavoring agents may be provided in the pharmaceutical composition.

In some embodiments, the polypeptide, the fusion protein, the nucleic acid or the vector may be comprised in a delivery particle, in particular, in combination with other natural or synthetic compounds, such as, e.g., lipids, protein, peptides, or polymers.

Within the scope of the invention said delivery particle is intended to provide, or “deliver”, the target cells, tissue or organ with the polypeptide, nucleic acid or nucleic acid vector according to the invention.

In some embodiment, the delivery particle may be in the form of a lipoplex, comprising cationic lipids; a lipid nano-emulsion; a solid lipid nanoparticle; a peptide-based particle; a polymer-based particle, in particular comprising natural and/or synthetic polymers; and a mixture thereof.

In some embodiment, a polymer-based particle may comprise a synthetic polymer, in particular, a polyethylene imine (PEI), a dendrimer, a poly (DL-Lactide) (PLA), a poly(DL-Lactide-co-glycoside) (PLGA), a polymethacrylate and a polyphosphoesters.

In some embodiment, the delivery particle further comprises at its surface one or more ligand(s) suitable for addressing the polypeptide, the nucleic acid or the nucleic acid vector to a target cell, tissue or organ.

In some embodiments, the pharmaceutical composition as described hereinabove is for use as a medicament.

In one embodiment, the polypeptide, fusion protein, pharmaceutical composition or medicament of the invention is for the treatment and/or prevention of a disease.

In one embodiment, the polypeptide, fusion protein, pharmaceutical composition or medicament of the invention is for the treatment and/or prevention of thrombopoietin-related diseases.

As used herein, the term “thrombopoietin-related diseases” refers to any disease or condition in which THPO contributes to, is involved in at any level, or promotes the onset or development of.

In some embodiments, thrombopoietin-related diseases of the invention include diseases or conditions associated with elevated blood levels of THPO. The elevated blood levels of THPO may be due to an abnormally high production (or overproduction) of THPO and/or to an abnormal low degradation of THPO.

In some other embodiments, thrombopoietin-related diseases of the invention include diseases or conditions associated with a hypersensitivity of TPOR to its ligand THPO. The hypersensitivity of TPOR may be due to an abnormally high activation (or overactivation) of TPOR. The overactivation of TPOR may be due to elevated blood levels of THPO. The overactivation of TPOR may be due to one or more mutation(s) in the gene encoding TPOR and/or JAK2. In one embodiment, the thrombopoietin-related disease is associated with or due to a constitutive activation of TPOR.

In some embodiments, thrombopoietin-related diseases include diseases or conditions where THPO-induced signaling accentuates pathological mechanisms affecting other signaling pathways.

In some embodiments, the polypeptide, fusion protein, pharmaceutical composition or medicament of the invention is for the treatment and/or prevention of conditions or diseases associated with elevated blood levels of THPO in a patient. In some embodiments, elevated blood levels of THPO means more than 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310 or 320 μg/mL. In some embodiments, elevated blood levels of THPO means more than 350, 400, 450, 500, 750 or 1000 μg/mL.

In some embodiments, thrombopoietin-related diseases are associated with elevated platelet count, wherein elevated platelet count means more than 150×10³, 200×10³, 250×10³, 300×10³, 350×10³, 400×10³, 450×10³ or 500×10³ platelets per μL of blood.

In some embodiments, thrombopoietin-related diseases include diseases or conditions associated with elevated blood levels of THPO and thus wherein THPO-induced signaling is increased. In some embodiments, the polypeptide, fusion protein, pharmaceutical composition or medicament of the invention is for the treatment and/or prevention of inflammatory conditions and infections associated with increased THPO-induced signaling.

In some embodiments, the polypeptide, fusion protein, pharmaceutical composition or medicament of the invention is for the treatment and/or prevention of thrombopoietin-related diseases selected from the group comprising or consisting of hematological diseases, chronic liver diseases, thrombopoietin-related infections, thrombopoietin-related inflammations and hereditary thrombocytosis, in a subject in need thereof.

In some embodiments, the polypeptide, fusion protein, pharmaceutical composition or medicament of the invention is for the treatment and/or prevention of diseases or conditions wherein TPOR is overactivated.

In one embodiment, the polypeptide, fusion protein, pharmaceutical composition or medicament is for the treatment and/or prevention of hematological diseases in a subject in need thereof.

In some embodiments, the polypeptide, fusion protein, pharmaceutical composition or medicament of the invention is for the treatment and/or prevention of hematological diseases selected from the group comprising or consisting of myeloproliferative neoplasms (MPN), de novo Acute Myeloid Leukemia (de novo AML), secondary Acute Myeloid Leukemia (secondary AML) and hereditary thrombocytosis, in a subject in need thereof, preferably myeloproliferative neoplasms (MPN) or de novo Acute Myeloid Leukemia (AML).

In a preferred embodiment, the polypeptide, fusion protein, pharmaceutical composition or medicament of the invention is for the treatment and/or prevention of myeloproliferative neoplasms (MPNs) in a subject in need thereof.

As used herein, MPN refers to a blood cancer type caused by a pathological constitutive activation of the JAK/STAT pathway that affects primarily hematopoietic stem cells and induces an abnormal expansion of cells of the myeloid lineage.

In one embodiment, the MPN is selected from the group comprising or consisting of essential thrombocythemia (ET), primary myelofibrosis (PMF) and polycythemia vera (PV).

In one embodiment, the polypeptide according to the invention alleviates, diminishes and/or suppresses THPO-induced signaling, and/or the JAK/STAT constitutive activation inducing the MPN.

In one embodiment, the polypeptide according to the invention alleviates, diminishes and/or suppresses THPO-induced signaling, and/or the JAK/STAT constitutive activation inducing the MPN in a trans action way.

In one embodiment, the polypeptide according to the invention comprises an amino acid sequence having at least 75% identity sequence with SEQ ID NO: 4 and alleviates, diminishes and/or suppresses THPO-induced signaling, and/or the JAK/STAT constitutive activation inducing the MPN.

In some embodiments, the polypeptide of the invention comprising an amino acid sequence having at least 75% identity sequence with SEQ ID NO: 4 has a better inhibition of the THPO-induced signaling, and/or the JAK/STAT constitutive activation inducing the MPN than the fragment region D1D2. In one embodiment, the polypeptide, fusion protein, pharmaceutical composition or medicament of the invention is for the treatment and/or prevention of de novo AML in a subject in need thereof.

In another embodiment, the polypeptide, fusion protein, pharmaceutical composition or medicament is for the treatment and/or prevention of hereditary thrombocytosis in a subject in need thereof. As used herein, hereditary thrombocytosis relates to a familial myeloproliferative disorder with clinical features resembling sporadic essential thrombocythemia, i.e., a disorder characterized by an excessive platelets production, that may be caused by mutations in the genes encoding THPO and/or TPOR.

In another embodiment, the polypeptide, fusion protein, pharmaceutical composition or medicament is for the treatment and/or prevention of chronic liver diseases in a subject in need thereof. In another embodiment, the polypeptide, fusion protein, pharmaceutical composition or medicament is for the treatment and/or prevention of thrombopoietin-related infections in a subject in need thereof. In another embodiment, the polypeptide, fusion protein, pharmaceutical composition or medicament is for the treatment and/or prevention of thrombopoietin-related inflammations in a subject in need thereof.

In some embodiments, the polypeptide, the fusion protein, the nucleic acid, the vector or the pharmaceutical composition of the invention is administered in combination with a further anticancer agent or with an anticancer vaccine.

In certain embodiments, the further anticancer agent or vaccine is to be administered in combination with, concomitantly or sequentially, the polypeptide, the fusion protein, the nucleic acid, the vector, the pharmaceutical composition or the medicament according to the invention.

In one embodiment, the further anticancer agent or vaccine is administered at the same time as the polypeptide, the fusion protein, the nucleic acid, the vector, the pharmaceutical composition or the medicament according to the invention of the invention (i.e., simultaneous administration optionally in a co-formulation). In one embodiment, the further anticancer agent or vaccine is administered at a different time than the polypeptide, the fusion protein, the nucleic acid, the vector, the pharmaceutical composition or the medicament according to the invention (i.e., sequential administration, where the further anticancer agent or vaccine is administered before or after the polypeptide is administered). In some embodiments, the further anticancer agent or vaccine may be administered in the same way as the polypeptide, the fusion protein, the nucleic acid, the vector, the pharmaceutical composition or the medicament according to the invention, or by using the usual administrative routes for that further anticancer agent or vaccine.

As used herein, the term “subject” refers to an animal, preferably a mammal, more preferably a human. In one embodiment, the subject is a man. In another embodiment, the subject is a woman. In one embodiment, the subject is a “patient”, i.e., the subject is awaiting the receipt of, or is receiving medical care, or was/is/will be the object of a medical procedure or treatment aiming to cure or treat the thrombopoietin-related disease, preferably a MPN, and/or alleviate the symptoms of the blood cancer, preferably a MPN. In some embodiments, the subject is monitored for the development of a thrombopoietin-related disease, preferably a MPN. In one embodiment, the subject is given a preventive treatment for a thrombopoietin-related disease, preferably a MPN. In one embodiment, the subject is an adult (for example a subject above the age of 18). In another embodiment, the subject is a child (for example a subject below the age of 18).

In some embodiments, the polypeptide, nucleic acid, vector or pharmaceutical composition according to the present invention is to be administered orally, parenterally, topically, by inhalation spray, rectally, nasally, buccally, vaginally or via an implanted reservoir. The term administration used herein includes subcutaneous, intravenous, intramuscular, intraocular, intra-articular, intra-synovial, intrasternal, intrathecal, intrahepatic, intralesional and intracranial injection or infusion techniques.

In a preferred embodiment, the polypeptide, nucleic acid, vector or pharmaceutical composition of the present invention is to be administered parenterally, subcutaneously, intravenously, or via an implanted reservoir.

In some embodiments, the polypeptide, nucleic acid, vector or pharmaceutical composition of the invention is in a form adapted for injection, such as, for example, for intraocular, intramuscular, subcutaneous, intradermal, transdermal or intravenous injection or infusion.

Examples of forms adapted for injection include, but are not limited to, solutions, such as, for example, sterile aqueous solutions, dispersions, emulsions, suspensions, solid forms suitable for using to prepare solutions or suspensions upon the addition of a liquid prior to use, such as, for example, powder, liposomal forms and the like.

The treatment may consist of a single dose or a plurality of doses over a period of time. The polypeptide, nucleic acid, vector or pharmaceutical composition according to the invention may be formulated in a sustained release formulation so as to provide sustained release over a prolonged period of time such as over at least 2 or 4 or 6 or 8 weeks. Preferably, the sustained release is provided over at least 4 weeks.

In certain embodiments, the effective amount of the polypeptide, nucleic acid, vector or pharmaceutical composition to be administered may depend upon a variety of parameters, including the material selected for administration, whether the administration is in single or multiple doses, and the subject's parameters including age, physical conditions, size, weight, gender, and the severity of the thrombopoietin-related disease to be treated.

In some embodiments, the polypeptide, nucleic acid, vector or pharmaceutical composition according to the invention is administered to the subject in need thereof in a therapeutically effective amount.

By “therapeutically effective amount”, it is meant a level or amount that is necessary and sufficient for slowing down or stopping the progression, aggravation, or deterioration of one or more symptoms of a thrombopoietin-related disease; or alleviating the symptoms of cancer; or curing a thrombopoietin-related disease, without causing significant negative or adverse side effects to the individual. In certain embodiments, an effective amount of the polypeptide according to the invention may range from about 0.001 mg to about 3,000 mg, per dosage unit, preferably from about 0.05 mg to about 1,000 mg, per dosage unit.

Within the scope of the instant invention, from about 0.001 mg to about 3,000 mg includes, from about 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,100 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg and 3,000 mg per dosage unit.

In certain embodiments, the polypeptide according to the invention is to be administered at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day.

In some embodiments, an effective amount of the nucleic acid or vector to be administered may range from about 1×10⁵ to about 1×10¹⁵ copies per dosage unit.

Within the scope of the instant invention, from about 1×10⁵ to about 1×10¹⁵ copies includes 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴ and 1×10¹⁵ copies, per dosage unit.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets. The formulations for use in the present invention may further include other agents conventional in the art having regard to the type of formulation in question, for example those suitable for oral administration may include flavoring agents.

Another object of the present invention is a kit (i) at least one polypeptide, at least one fusion protein, at least one nucleic acid, at least one vector, at least one pharmaceutical composition or at least one vaccine according to the invention, and (ii) means to administer said polypeptide, fusion protein, nucleic acid, vector, pharmaceutical composition or medicament. In one embodiment, the kit is for treating and/or preventing a disease, preferably a thrombopoietin- or thrombopoietin receptor related disease, more preferably a MPN.

In certain embodiments, the means to administer the polypeptide, the fusion protein, the nucleic acid, the vector, the pharmaceutical composition or the medicament according to the invention may include a syringe, a trocar, a catheter, a cup, a spatula, and the likes.

In one embodiment, the kit further comprises an anticancer agent or vaccine, preferably for treating a MPN.

In some further aspects, the invention also relates to the use of the polypeptide, the fusion protein, the nucleic acid, the vector, the pharmaceutical composition or the medicament according to the invention for the manufacture or the preparation of a medicament.

The invention also relates to a polypeptide, fusion protein, nucleic acid, vector, pharmaceutical composition or the medicament according to the invention, for the manufacture of a medicament for treating and/or preventing thrombopoietin-related diseases, preferably selected from the group comprising or consisting of myeloproliferative neoplasms (MPN), hematological diseases, hereditary thrombocytosis, de novo AML (Actute Myeloid Leukemia), chronic liver diseases, thrombopoietin-related infections and thrombopoietin-related inflammations, preferably MPN.

The invention also relates to a method for treating and/or preventing thrombopoietin-related diseases, preferably selected from the group comprising or consisting of myeloproliferative neoplasms (MPN), hematological diseases, hereditary thrombocytosis, de novo AML (de novo Acute Myeloid Leukemia), secondary Acute Myeloid Leukemia (secondary AML), chronic liver diseases, thrombopoietin-related infections and thrombopoietin-related inflammations, preferably MPN, in a subject in need thereof comprising administering to said subject a therapeutically effective amount of the polypeptide, fusion protein, nucleic acid, vector, pharmaceutical composition or medicament according to the invention.

The present invention also relates to a polypeptide, fusion protein, nucleic acid, vector, pharmaceutical composition or medicament according to the invention, for use in inhibiting the expansion of cancer cells.

The present invention also relates to a method for inhibiting the expansion of cancer cells in an individual in need thereof, comprising at least the step of administering to the individual a therapeutically effective amount of polypeptide, fusion protein, nucleic acid, vector, pharmaceutical composition or medicament according to the invention.

The present invention also concerns a polypeptide, fusion protein, nucleic acid, vector, pharmaceutical composition or medicament according to the invention, for use in improving the overall survival of an individual having cancer.

The present invention also concerns a method for improving the overall survival of an individual having cancer, comprising at least the step of administering to the individual a therapeutically effective amount of polypeptide, fusion protein, nucleic acid, vector or pharmaceutical composition according to the invention.

The present invention also concerns a polypeptide, fusion protein, nucleic acid, vector, pharmaceutical composition or medicament according to the invention, for use in improving the prognosis of an individual having cancer.

The present invention also concerns a method for improving the prognostic of an individual having cancer, comprising at least the step of administering to the individual a therapeutically effective amount of polypeptide, fusion protein, nucleic acid, vector, pharmaceutical composition or medicament according to the invention.

Another object of the present invention is a method of inhibiting or reducing or preventing or disturbing the binding of THPO to TPOR in an individual, comprising at least the step of administering to the individual an effective amount of polypeptide, fusion protein, nucleic acid, vector, pharmaceutical composition or medicament according to the invention. In one embodiment, inhibiting or reducing the binding of THPO to TPOR inhibits or reduces or prevents or disturbs TPOR signaling in said subject. In one embodiment, inhibiting or reducing or preventing or disturbing the binding of THPO to TPOR inhibits the activation of TPOR in said subject. In one embodiment, inhibiting or reducing or preventing or disturbing the binding of THPO to TPOR treats or prevents thrombopoietin-related diseases.

The present invention also relates to a method of competing endogenous THPO of an individual with a polypeptide or fusion protein of the invention, comprising at least the step of administering to the individual an effective amount of polypeptide, fusion protein, nucleic acid, vector, pharmaceutical composition or medicament according to the invention. In one embodiment, competing endogenous THPO inhibits or reduces or prevents or disturbs TPOR signaling in said subject. In one embodiment, competing endogenous THPO inhibits the activation of TPOR in said subject. In one embodiment, competing endogenous THPO treats or prevents thrombopoietin-related diseases.

A further object of the present invention is a method of inhibiting or reducing or preventing or disturbing TPOR signaling or TPOR-dependent signaling in an individual, comprising at least the step of administering to the individual an effective amount of polypeptide, fusion protein, nucleic acid, vector, pharmaceutical composition or medicament according to the invention. Another object of the present invention is a method of inhibiting reducing or preventing or disturbing the activation of TPOR in an individual, comprising at least the step of administering to the individual an effective amount of polypeptide, fusion protein, nucleic acid, vector, pharmaceutical composition or medicament according to the invention. In one embodiment, inhibiting or reducing or preventing or disturbing TPOR signaling or TPOR-dependent signaling treats or prevents thrombopoietin-related diseases.

### BRIEF DESCRIPTION OF THE DRAWINGS

FIGS. 1A to 1B are graphs showing THPO trapping by soluble extracellular domain (ECD)-TPOR and Fc-coupled soluble ECD-TPOR in vitro. Soluble ECD-TPOR (A) and soluble ECD-TPOR-Fc fusion proteins (B) were collected after their secretion by HEK293T in culture. They were incubated with human recombinant THPO and were then added to the culture medium of Baf3 expressing TPOR at their cell surface. The growth of cells incubated with soluble ECD-TPOR species (A) or with soluble ECD-TPOR-Fc fusion proteins (B) was measured by daily cells count during five days, in presence (hatched lines) or in absence (continuous lines) of THPO. The growth of cells was measured in presence of soluble D1+/−Fc (deep grey, triangle marks), of soluble D1D2+/−Fc (light grey, round marks), D1D2D3D4+/−Fc (black, square marks), and compared with the cell's growth in absence of the soluble ECD-TPOR (black, round marks).

FIGS. 2A to 2B are histograms showing the dosage of circulating THPO by ELISA from MPL knock out mice before and after exposition to soluble TPOR in vivo. MPL−/− mice were injected subcutaneously with MEFs cells transduced by retroviral particles (pMEGIX vector) expressing the murine soluble D1D2D3D4 fused with Fc (soluble ECD-TPOR-Fc). After 2 weeks post-transplant of MEFs cells, a vascularized mass of cells under the skin grew, and 2 successive bleedings with an interval of 1 week were performed: a first bleeding (bleeding 1) at 3 weeks after post-transplant (A), and a second bleeding (bleeding 2) at 4 weeks post-transplant (B). The graphics show the concentration of circulating THPO in blood for each group of mice (5-6 mice per group). * represents significant difference p<0.05; ** represents significant difference p<0.01; *** represents significant difference p<0.001 (one-way ANOVA, Holm-Sidak's multiple comparisons test), ns: not significant.

### EXAMPLES

The present invention is further illustrated by the following examples.

### Example 1: In Vitro THPO Trapping by Soluble TPOR Extracellular Domain Fused or not with Fc

### Materials and Methods

Soluble extracellular domain (ECD) species TPOR, namely D1, D1D2 and D1D2D3D4, and -Fc fusion proteins of these ECD species were collected after their secretion by cultured HEK293T cells. They were incubated with human recombinant THPO to allow the interaction between the soluble ECD-TPOR (or the soluble ECD-TPOR-Fc fusion proteins) and THPO, and they were added to the culture medium of hematopoietic cell line Baf3 cells stably expressing human TPOR at their surface, whose proliferation is strictly dependent on cytokines. The cell growth was measured by daily cells count during five days to probe the ability of soluble ECD-TPOR and ECD-TPOR-Fc to sequestrate THPO in the culture medium.

### Results

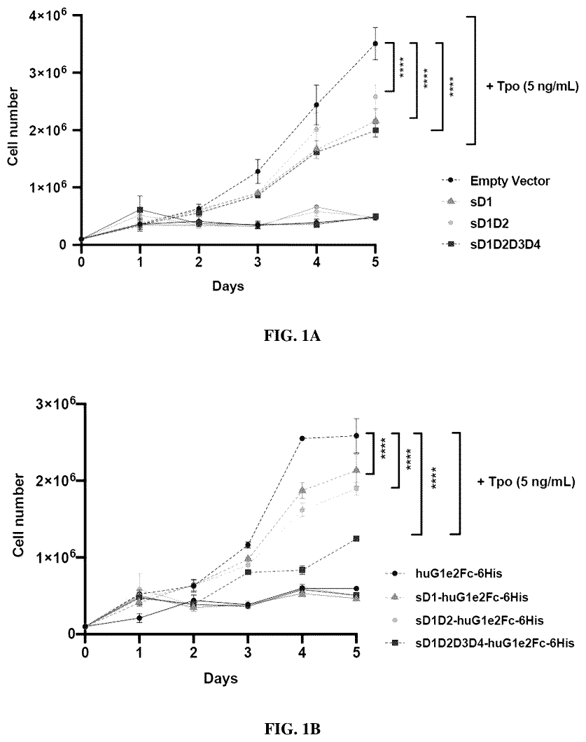

Results revealed a significant inhibition of proliferation of Baf3 cells in presence of either one of the soluble ECD of TPOR (D1, D1D2 and D1D2D3D4) (FIG. 1A) as wells as with Fc-coupled soluble species of the receptor (FIG. 1B).

Notably, THPO inhibition was most efficient with the full ECD of TPOR (D1D2D3D4) than with truncated forms (D1 and D1D2) (FIG. 1A). This difference in inhibition potency was markedly increased by the coupling of the soluble D1D2D3D4 to the Fc fragment (FIG. 1B) compared to D1-Fc and D2-Fc.

### Example 2: In Vivo Validation of THPO Signaling Inhibition by Soluble D1D2D3D4 of TPOR Fused with Fc

### Materials and Methods

MPL−/− mice, i.e., mice constitutively deficient for TPOR expressing very high plasmatic concentration of thrombopoietin (as it is not degraded by its receptor), were injected subcutaneously with mouse embryonic fibroblasts (MEFs) cells transduced by retroviral particles (pMEGIX vector) containing DNA encoding the murine soluble D1D2D3D4 of TPOR fused to Fc (5-6 mice per group) to allow their production by muscle cells and release in blood circulation. After 2 weeks post-transplant of MEFs cells, a vascularized mass of cells under the skin grew and 2 successive bleedings with an interval of 1 week were performed: a first bleeding at 3 weeks after post-transplant, and a second bleeding at 4 weeks post-transplant.

### Results

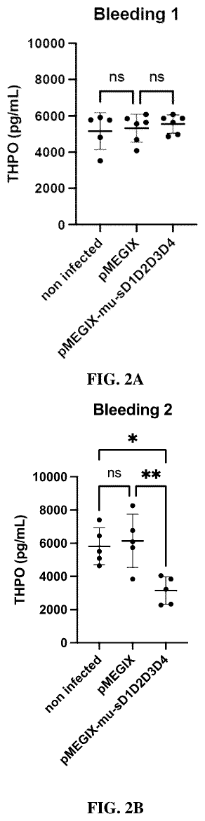

The first bleeding at 3 weeks after the transplant of MEF cells does not yield a significant difference between transplanted and untransplanted mice (FIG. 2A). However, in the second bleeding one week later (4 weeks after transplant), measurement of plasmatic THPO showed that the Fc-coupled soluble D1D2D3D4 was able to significantly decrease the circulating levels of THPO (FIG. 2B). This results thus demonstrates that D1D2D3D4-Fc acts as a trap molecule for circulating THPO in vivo, thereby validating in vitro results.

1-16. (canceled)

17. A method for treating and/or preventing a disease, in a subject in need thereof comprising administering to said subject a therapeutically effective amount of a polypeptide comprising an amino acid sequence having at least 75% sequence identity with SEQ ID NO: 2 or a variant thereof that binds to thrombopoietin (THPO);
a fusion protein comprising said polypeptide;
a nucleic acid comprising a sequence encoding said polypeptide or said fusion protein;
a vector comprising said nucleic acid; or
a composition comprising said polypeptide, said fusion protein, said nucleic acid, or said vector, and at least one pharmaceutically acceptable vehicle.

18. The method according to claim 17, wherein said polypeptide is capable of inhibiting, reducing, preventing and/or disturbing the binding of thrombopoietin to its receptor (TPOR/MPL).

19. The method according to claim 17, wherein said polypeptide is capable of inhibiting, reducing, preventing and/or disturbing the TPOR signaling pathway or TPOR-dependent signaling pathway.

20. The method according to claim 17, wherein said amino acid sequence has at least 75% sequence identity with SEQ ID NO: 3.

21. The method according to claim 17, wherein said amino acid sequence has at least 75% sequence identity with SEQ ID NO: 4.

22. The method according to claim 17, wherein said fusion protein further comprises a second polypeptide that increases stability and/or immunogenicity of the polypeptide.

23. The method according to claim 17, wherein said fusion protein further comprises a Fc region of an immunoglobulin or a functional equivalent thereof.

24. The method according to claim 23, wherein said immunoglobulin is IgA, IgD, IgE, IgG, or IgM.

25. The method according to claim 23, wherein said immunoglobulin is IgG.

26. The method according to claim 17, wherein the disease is a thrombopoietin-related disease selected from the group consisting of hematological diseases, chronic liver diseases, thrombopoietin-related infections, thrombopoietin-related inflammations and hereditary thrombocytosis.

27. The method according to claim 17, wherein said thrombopoietin-related disease is selected from the group consisting of myeloproliferative neoplasms (MPN), de novo AML (Acute Myeloid Leukemia).

28. The method according to claim 17, wherein said thrombopoietin-related disease is a myeloproliferative neoplasm (MPN).

29. A kit for treating and/or preventing thrombopoietin-related diseases comprising (i) a polypeptide comprising an amino acid sequence having at least 75% sequence identity with SEQ ID NO: 2 or a variant thereof that binds to thrombopoietin (THPO), a fusion protein said polypeptide, or a pharmaceutical composition comprising said polypeptide, said fusion protein, said nucleic acid, or a vector comprising said acid nucleic, and at least one pharmaceutically acceptable vehicle, and (ii) means to administer said polypeptide, fusion protein or pharmaceutical composition, optionally further comprising an anticancer agent or vaccine.

30. A method for inhibiting the expansion of cancer cells in an individual in need thereof, comprising at least the step of administering to the individual a therapeutically effective amount of
a polypeptide comprising an amino acid sequence having at least 75% sequence identity with SEQ ID NO: 2 or a variant thereof that binds to thrombopoietin (THPO);
a fusion protein comprising said polypeptide;
a nucleic acid comprising a sequence encoding said polypeptide or said fusion protein;
a vector comprising said nucleic acid; or
a composition comprising said polypeptide, said fusion protein, said nucleic acid, or said vector, and at least one pharmaceutically acceptable vehicle.
